(19) United States
(12) Patent Application Publication
(10) Pub. No.: US 2024/0081662 A1
(43) Pub. Date: Mar. 14, 2024

(54) Transformation of Heart-Motion-Induced Signals Into Blood Pressure Signals

(71) Applicant: Fraunhofer Gesellschaft zur Förderung der angewandten Forschung e.V., München (DE)

(72) Inventors: Marian Häscher, Rostock (DE); Florian Höpfner, Rostock (DE); Helmut Tödtmann, Rostock (DE)

(21) Appl. No.: 18/513,648

(22) Filed: Nov. 20, 2023

Related U.S. Application Data
(63) Continuation of application No. PCT/EP22/63773, filed on May 20, 2022.

(30) Foreign Application Priority Data
May 20, 2021 (DE) 102021205185

(51) Int. Cl. A61B 5/021; A61B 5/00; A61B 5/11

(52) U.S. Cl. CPC A61B 5/021; A61B 5/1102; A61B 5/7221; A61B 5/7257; A61B 5/7264; A61B 5/7267; A61B 5/742; A61B 2503/40

(57) ABSTRACT

Disclosed is a method for generating an ABP signal, with at least one heart-motion-induced signal being detected. The at least one detected heart-motion-induced signal is transformed into at least one ABP signal. The transformation is carried out using a model that was generated by machine learning. The heart-motion-induced signal constitutes the input value, and the ABP signal constitutes the output value of the transformation.

### CROSS-REFERENCE TO RELATED APPLICATIONS

This application is a continuation of International Application No. PCT/EP2022/063773 filed May 20, 2022, which claims the priority to German Application No. DE102021205185 filed May 20, 2021. The entire disclosures of the above applications are incorporated by reference.

### FIELD

The invention relates to a method and a system for determining an ABP signal and a computer program product.

### BACKGROUND

The blood pressure is an important indicator for medical diagnoses, for example in case of hypertension-related diseases. In numerous cases, it may be required or desirable to detect a continuous blood pressure signal, namely, a so-called aortic blood pressure signal or ABP signal for diagnostic purposes. Currently, such a continuous blood pressure signal is usually recorded invasively. In known methods, for example, a cannula or a catheter is introduced into a blood vessel to then continuously detect the blood pressure. Further, also conventional, non-invasive measuring devices for measuring the blood pressure such as, e.g., upper arm cuff devices are known. However, these can only perform measurements at individual points in time, i.e., a discrete and not a continuous measurement. Moreover, these devices have to mechanically interrupt or at least reduce the blood flow. Furthermore, the measurement procedure is prone to errors, e.g., by inflating the cuff since the cuff has to be applied to a firm point of the body, e.g., about 1-2 cm above of the arm bend on the upper arm. Likewise, it has to be ensured that the cuff sits neither too tight nor excessively loose. Further, it was observed that a repeated measurement without a sufficient pause may result in falsified measuring values which further reduces the suitability for a continuous blood pressure detection.

Likewise, these known methods are also contact based which, in some cases, means an increased risk of infection on the part of the patient. In addition, the known approach may be disagreeable or even painful for some patients.

Other known approaches make use of a plurality of measuring systems such as, e.g., ECG devices or SCG detection means and PPG devices for measuring the so-called pulse wave transit time also referred to as pulse transit time, PTT, between two points, e.g., the heart and the finger. The pulse wave transit time renders the subsequent determination of the blood pressure possible. However, this approach requires the combination of various measuring systems and measurement methods which is, on the one hand, elaborate, but on the other hand, also imprecise. Likewise, such methods depend on a previous, person-specific calibration prior to each measurement for performing a conventional measurement of the blood pressure.

Other known approaches make use of PPG signals for performing a measurement of the blood pressure.

Further, the detection of seismocardiography signals (SCG signals) is known which can also be referred to as precordial motion signals. Here, the precordium may refer to a part of the chest wall in front of the heart. Therefore, the precordial motion signal may include information about the movement of this part of the chest wall. Particularly, such a signal includes information about movements, particularly vibrations, of the precordium caused by cardiac motion. Based on such signals, even movements of heart valves, e.g., the aortic valve or the mitral valve can be detected, and associated characteristics can be identified. While the electric stimuli made visible in the ECG examination represent the electric stimuli occurring prior to each muscular movement within the heart cycle, the SCG signal represents the resulting movements measured at the precordial position. In this approach, for example, widely used inertial sensors such as acceleration sensors or gyroscopes are used. However, pressure or radar sensors may also be used.

Likewise, the detection of phonocardiography signals is known, these being audio signals generated by receiving sound waves, the sound waves being caused by cardiac motion.

Likewise, the detection of ballistocardiography signals is known, these detecting the resonance of the entire body caused by the cardiac motion. Ballistocardiograms may be detected on the entire body and are therefore not limited to a specific measuring point.

A major part of research in the field of mobile and portable seismocardiography concentrates on the extraction of vital parameters such as the heart frequency, the heart frequency variability, or the respiration frequency. Even though these parameters provide for valuable information about the medical condition of the users it is desirable to continuously detect a blood pressure to extend the diagnostic possibilities of a physician.

Further, methods of machine learning are known, also in cardiology. A plurality of known methods uses convolutional autoencoders to compress health data by reducing the complexity or the noise in biological signals as shown for EEG and ECG signals.

Apart from the use of neural networks for analyzing ECG data, there is a number of publications applying machine learning to signals from other types of sensors. CNNs (convolutional neural networks) can be used for estimating the heart frequencies of PPG sensors (photoplethysmography sensors) or for the automatic identification of cardiovascular disorders from SCG data.

From prior art, WO2020/009387 A1 discloses a method and a device for estimating the segmental blood pressure using a circular neural network. The document teaches that a biometric signal is detected and analyzed, however, while characteristic information is extracted, and a blood pressure parameter is calculated based on the characteristic information. Moreover, a blood pressure for a future point in time is determined by a neural network, an input value for this determination being a calculated blood pressure.

Further, US2019/274552A1 describes a cuffless determination of a blood pressure. For this purpose, a blood pressure monitor comprises a processor which extracts blood pressure-related characteristics from a BCG signal and estimates a blood pressure based on at least part of the extracted characteristics. Here, the blood pressure estimator may be determined by methods of machine learning.

Further, US2020/330050A1 discloses health monitoring systems. Particularly, it is disclosed that output signals of acceleration sensors are supplied to a “peak pattern detector” which in turn provides for input signals for a blood pressure estimator.

These documents teach a characteristic extraction from a detected signal as an important feature for the determination of the blood pressure, the extracted characteristics then constituting input values for a method for determining/estimating the blood pressure. For example, US2020/330050A1 teaches a peak value detection and estimation as an important feature, the points in time of these peak values then constituting input values for a non-linear regression model estimating the blood pressure.

US2019/274552A1 teaches an extraction of blood pressure-related characteristics from a BCG signal which are then used as the basis for a blood pressure estimation as an important feature. WO2020/009387 A1 teaches as important that a characteristic is extracted from a biosignal, and then a past blood pressure-related parameter is calculated based on the extracted characteristic. Then, a future blood pressure is determined by using the blood pressure-related parameter as an input value for a neural network. This characteristic extraction is elaborate and prone to errors and may reduce the quality of the estimated blood pressure signal. Further, the characteristic determination requires additional computational effort.

Likewise known is the document M. S. Imtiaz et al., “Correlation between seismocardiogram and systolic blood pressure,” 2013 26th IEEE Canadian Conference on Electrical and Computer Engineering (CCECE), Regina, SK, Canada, 2013, pp. 1-4, doi: 10.1109/CCECE.2013.6567773, which analyzes a correlation between seismocardiogram and blood pressure.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Therefore, the technical problem arises to provide a method and a system for generating a particularly continuous ABP signal as well as a computer program product rendering an easy, precise, and reliable generation of the ABP signal possible which requires as little computational effort as possible, particularly an invasive detection involving the drawbacks described above being avoided.

The solution to the technical problem follows from the subject matters having the features of the independent claims. Further advantageous implementations of the invention follow from the subject matters having the features of the dependent claims.

What is proposed is a method for generating an ABP signal which may refer to an aortic blood pressure signal or an arterial blood pressure signal, at least one heart-motion-induced signal being detected. A heart-motion-induced signal may refer to a signal caused by cardiac motion. It is also possible that a plurality of heart-motion-induced signals is detected, particularly also signals of various types. This will be explained below. Particularly, the ABP signal is a continuous ABP signal. This may mean that a chronological progression of the ABP signal is generated, particularly a wave-shaped timeline. Particularly, the continuous aortic blood pressure signal defines the aortic blood pressure, and the continuous arterial blood pressure signal a blood pressure in an artery for each point in time of a predetermined determination period.

Particularly, a heart-motion-induced signal may be an SCG signal (a seismocardiography signal), or a PCG signal (a phonocardiography signal), or a BCG signal (a ballistocardiography signal). This heart-motion-induced signal may be generated by an appropriate detection means. For example, the SCG signal may be generated by an appropriate SCG detection means, the PCG signal by an appropriate PCG detection means, and the BCG signal by an appropriate BCG detection means. However, the heart-motion-induced signal is particularly not an ECG signal, particularly since the ECG signal is the signal which induces the heart motion (and not the other way around).

Such an SCG detection means may comprise, for example, at least one acceleration sensor, e.g. a MEMS acceleration sensor, particularly a MEMS gyroscope, or a radar sensor, particularly a Doppler radar sensor. As explained above, the SCG signal includes or encodes information about the cardiac motion. Such acceleration sensors may be uniaxial or triaxial piezoelectric acceleration sensors or MEMS acceleration sensors, triaxial MEMS acceleration sensors or gyroscopes, laser Doppler vibrometers, microwave Doppler radar sensors, or a so-called Airbourne ultrasound surface motion camera (AUSMC). A PCG detection means may particularly comprise a microphone, particularly a microphone of a mobile end device such as, e.g., a mobile telephone, or a laser microphone. A BCG detection means may comprise, e.g., at least one pressure sensor, e.g., a pressure sensor implemented as a load cell.

Further, the at least one detected heart-motion-induced signal is transformed into at least one ABP signal. Transformation processes will be explained in more detail below. It is—as explained in more detail below—also possible that a plurality of detected heart-motion-induced signals is transformed into an ABP signal.

For example, it was surprisingly found that a heart-motion-induced signal and an ABP signal possess a comparable information content with regard to the heart activity since ABP signals also include or encode information about cardiac motion since the aortic blood pressure is influenced by cardiac motion. In the reverse, therefore, a heart-motion-induced signal also includes information about mechanical activities of the heart.

Since heart-motion-induced signals are regularly incomprehensible for the user without appropriate processing because they are usually not particularly used for a diagnosis in daily hospital and practice routine and their interpretation is usually not part of the training of a physician an ABP signal which is usually informative for a larger group of persons can be generated by the transformation so that the medical applicability, e.g., for diagnostic purposes increases.

Likewise, advantageously, mechanically contacting the patient or an invasive detection are not absolutely indicated for the detection of heart-motion-induced signals.

The heart-motion-induced signal is therefore detected in a contact-free manner, i.e., without mechanically contacting a patient using an associated sensor. For example, this may take place by the detection means being positioned at a distance to the patient, for example, in a mattress on which the patient rests, or in a seat in which the patient sits. If the detection means comprises, e.g., a radar sensor it is only required to position the detection means so that the patient or a chest area of the patient is located in the detection range of the radar sensor.

However, it is also possible that the heart-motion-induced signal is detected by a sensor which mechanically contacts the patient, or which is disposed in or on the patient for the detection. For example, it is possible that the detection means is integrated in a pacemaker, particularly a rate adaptive pacemaker. A pacemaker may comprise such a detection means, particularly a detection means implemented as an acceleration sensor to adapt a rhythm of a patient's heartbeat depending on the signal detected by the detection means, e.g., to adjust it to the current motion state as well as the pulse requirements. In order to render this possible, activities are identified depending on output signals of the acceleration sensors, and the rhythm of the heartbeat is increased correspondingly, e.g., in case of increased strain (e.g., in case of a change from walking to climbing stairs). The acceleration sensors used for this purpose may also be used to detect a heart-motion-induced signal.

A signal detected by such a detection means may then be transmitted to, e.g., a calculating means, e.g., in a wireless manner using appropriate methods of data transmission, the calculating means then carrying out the transformation. This (external) calculating means may be, e.g., a calculating means of a mobile end device. Alternatively, it is conceivable that the pacemaker comprises a calculating means which will then carry out the transformation. Such a calculating means of the pacemaker may be integrated in the same in the form of an embedded system. For example, the calculating means may be implemented as an integrated circuit specifically designed for performing the transformation. This integrated circuit may, e.g., provide for the functionality of a neural network.

The use of a detection means which is integrated in a cardiac pacer advantageously renders the utilization of already existing sensors disposed close to the heart possible which results in a good signal quality of the heart-motion-induced signals. This in turn improves the measuring accuracy and therefore also the accuracy of the ABP signal generated according to the invention. Moreover, also a convenient certification of a system for generating an ECG signal as a medical product comprising the detection means of the cardiac pacer is rendered possible due to the extended utilization of an already certified cardiac pacer.

With the transformation, therefore, the at least one heart-motion-induced signal which represents, e.g., precordial movements, sound waves caused by these movements, or movements of the entire body is transformed into a signal representing or reproducing the chronological progression of the aortic blood pressure.

The transformation into an ABP signal is a direct transformation. The transformation may also include a plurality of partial transformations, the heart-motion-induced signal being transformed into an intermediate signal, e.g., in a first partial transformation, and the intermediate signal being transformed into the ABP signal in a further partial transformation. Of course, it is also possible that more than two partial transformations are carried out.

The proposed method advantageously results in a simple and reliable generation of an ABP signal which particularly, but not necessarily, takes place in a contactless fashion, however, in any case in a non-invasive manner. Therefore, the proposed method renders a reliable long-term recording of ABP signals possible, particularly over a period of more than 24 hours because heart-motion-induced signals can be recorded over such a period of time without problems and then be transformed, particularly since the generation takes place in a non-invasive manner.

Further, it is possible to advantageously implement and thus to retrofit the claimed method in existing devices including a detection means suitable for detecting heart-motion-induced signals so that these devices are enabled to generate an ABP signal. For example, mobile telephones usually comprise acceleration sensors. These can be made use of to generate SCG signals, for example, by placing a mobile telephone on a patient's chest and detecting output signals of the acceleration sensor. These output signals may then be transformed into an ABP signal by the proposed transformation. For generating PCG signals, also a microphone of a mobile telephone can be used.

According to the invention, the transformation is performed using a model generated by machine learning.

Further, the heart-motion-induced signal constitutes the input value, and the ABP signal the output value of the transformation. Particularly, no extraction of characteristics from the heart-motion-induced signal takes place which will then constitute input values of the transformation. Therefore, it is possible that an unprocessed heart-motion-induced signal or a filtered heart-motion-induced signal constitutes the input value of the transformation, the filtering not serving a characteristic extraction. Further, the transformation particularly does not comprise a step for determining predetermined characteristics. The heart-motion-induced signal may also constitute the only input value of the transformation. In other words, apart from the heart-motion-induced signal, no other input value is considered in the transformation.

Here, the term machine learning includes or refers to methods for defining the model based on training data. For example, it is possible to define the model by methods of supervised learning, the training data, i.e., a training data set comprising input data and output data to this end. As the input data, heart-motion-induced signals may be provided here, the ABP signals corresponding to these heart-motion-induced signals being provided as the output data.

Particularly, input and output data of such training data may be generated by simultaneously generating heart-motion-induced signals and ABP signals, these simultaneously generated data then constituting the input and output data for the training. Methods and a device for simultaneously generating such data are known from the prior art explained in the introduction to the description. For example, the model may learn the interrelation of the seismocardiogram, the ballistocardiogram, or the phonocardiogram and the blood pressure signal here. Such methods of supervised learning are known to the person skilled in the art. It is also conceivable that methods of unsupervised learning are used for defining the model. For generating training data, e.g., continuous aortic blood pressure signals (ABP signals) and, simultaneously, seismocardiography signals (SCG signals) may be recorded.

For example, in a first step, simultaneously, an invasive, continuous detection of the ABP signal and a continuous detection of a heart-motion-induced signal, particularly of an SCG signal, may be performed on members of a first group of test persons. Here, the ABP signal may be directly measured in a blood vessel of a test person.

Then, a training of a first model for transforming the heart-motion-induced signal into the ABP signal based on the data of the group of test persons detected in this way and the amplitudes available in this sample, particularly of the amplitudes of systole and diastole will follow. The training data used for the model generation are referred to as the first training data.

Subsequently, further training data may be generated by non-invasively detecting a blood pressure signal at discrete points in time, i.e. not continuously, and, simultaneously and continuously, a heart-motion-induced signal on members of another group of test persons. The time-discrete measurement of the blood pressure may be carried out, for example, using an upper arm cuff measuring device. Then, the transformation of the heart-motion-induced signal of the test persons of the other group of test persons into an ABP signal may be carried out using the first model, however, the ABP signal determined by the transformation, particularly the amplitudes of systole and diastole, being subsequently corrected based on the time-discrete measuring values, particularly so that a deviation of the amplitudes determined by the transformation from the amplitudes measured in the time-discrete measurement is minimal. This correction is advantageous because the first training data set may potentially not include all variants of the amplitude values (due to the clinical laboratory conditions, e.g. lying down without activity, possibly sedation).

The ABP signals determined by transformation and corrected in this way and the heart-motion-induced signals of the other group of test persons then constitute another training data set. For example, it is possible that the training of an updated model is carried out based on the entirety of the first and the other training data set, whereat the entirety may also be referred to as a fused training data set. Thus, an extension of the training data set is rendered possible without further invasive measurements being required. This extension of the training data set can also be repeated in an easy manner to expand the training data set.

After the preparation of the model, i.e., after the training phase, the model parametrized in this way may be used in the so-called inference phase to then generate the ABP signals to be determined from input data in the form of heart-motion-induced signals, i.e., to perform the proposed transformation. This results in a reliable and high-quality generation of ABP signals.

It is possible that the model is determined in a user- or patient-unspecific and/or detection means-unspecific manner, the model determined in this way then being used to carry out the transformation for a specific user and/or a specific detection means. This may mean that the model is not individually determined for a specific user and/or for a specific detection means but can then be used for an individual user and/or an individual detection means in the inference phase. It is therefore possible that the model does not have to be newly trained for each user and/or each detection means. It may particularly be trained once, using an appropriately large data set (training phase), and then used independent of the user and/or the detection means as a model, e.g., for all users (inference phase). This advantageously results in that an applicability of the method is improved, particularly since no specific training has to take place for each user and/or each detection means. For example, the same model can be used for transforming signals generated by different detection means.

Here, the appropriate data set includes data generated for at least a predetermined number of different ill or healthy persons and/or for at least a predetermined number of physiologies and/or for at least a predetermined number of different diseases.

However, it may of course be required to train the model with the aid of input data having the same characteristic, i.e., only with SCG signals, PCG signals, or BCG signals, in which case, however, various detection means or various configurations of a detection means can be used for detecting these signals having the same characteristic. However, it is of course also possible that the model is determined in a user- and/or detection means-specific manner.

Suitable mathematic algorithms for machine learning include: decision tree based methods, ensemble methods—(e.g., boosting-, random forest-)based methods, regression-based methods, Bayesian methods—(e.g. Bayesian belief networks-)based methods, kernel methods—(e.g., support vector machines-)based methods, instance—(e.g., k-nearest neighbor-)based methods, association rule learning-based methods, Boltzmann machine-based methods, artificial neural networks—(e.g., perceptron-)based methods, deep learning—(e.g., convolutional neural networks-, stacked autoencoders-)based methods, dimensionality reduction-based methods, regularization methods-based methods.

For training, e.g., a neural network, regularly a large amount of training data is required to ensure a desired quality of the transformation. The amount of training data may depend on factors such as the complexity of the underlying problem, the required accuracy, and the aspired adaptability of the network to be trained. The field of application, i.e., the domain in which the network is to be deployed is frequently the most important element in the determination of these factors and therefore the determination of the amount of training data. With adequate prior knowledge of the domain, it is possible to prepare data which bring about a faster convergence to the optimum solution or render such convergence possible in the first place for the training of the network, and to thereby reduce the required amount of training data.

The proposed method is employed in a medical environment. Therefore, a high accuracy is desirable. Added up to this is a comparably high complexity since ABP signals and heart-motion-induced signals are different from each other due to the different sensorics for their detection. However, this usually results in a large amount of data for training a neural network. A possible step for reducing the required amount of data is filtering the training data, particularly the input data and/or the output data. Particularly, input and output data of a training data set may be generated by simultaneously generating heart-motion-induced signals and ABP signals and then filtering them prior to the training. In this way, both the memory requirements and the required computing time and/or capacity for the determination/generation of the model are reduced. For example, it is possible to filter the training data using a filter, particularly a bandpass filter, e.g., a Butterworth filter to attenuate high-frequency as well as low-frequency components in the training data. For example, a first, lower cutoff frequency of a bandpass filter may be 0.5 Hz, and another, upper cutoff frequency may be 200 Hz. Likewise, conceivable is the utilization of high- and/or low-pass filters or other filters (e.g., polynomial filters) which filter the relevant undesired frequencies out of the training data. Alternatively, however, the generated signals may also be used for the training unfiltered.

In another embodiment, the at least one heart-motion-induced signal is an SCG signal. This advantageously results in a reliable provision of an ABP signal since SCG signals can be reliably generated. Further, it advantageously follows that a SCG signal has a broad frequency spectrum (particularly a broader frequency spectrum as compared to a BCG signal) and thus a high information density and can be generated in a contactless manner. Particularly, an SCG signal may include information about a heart valve movement. Advantageously, it also follows that a SCG signal, particularly as compared to a BCG signal, may include fewer motion artefacts, particularly since it includes, in comparison, components of a higher frequency. These properties, in turn, result in a high signal quality. Likewise, it has been found that determining the model using SCG signals is possible at a sufficiently rapid convergence.

Alternatively, the heart-motion-induced signal is a PCG signal. As it comprises a broad frequency spectrum (particularly a broader frequency spectrum as compared to the SCG signal and a BCG signal) this advantageously results in a precise generation of an ABP signal. Therefore, the PCG signal also has a high information density. Alternatively, the heart-motion-induced signal is a BCG signal. As it can be measured on the entire body this advantageously results in a flexible detection and therefore generation of an ABP signal. It is conceivable that a plurality of particularly different heart-motion-induced signals is detected, e.g., a plurality of SCG signals, a plurality of BCG signals, or a plurality of PCG signals. Likewise, at least two different signals of the signal set comprising SCG, PCG, and BCG signals may be detected, the at least one ABP signal then being generated by transforming these different signals into the at least one ABP signal. It is also conceivable that a fused heart-motion-induced signal is generated from the different heart-motion-induced signals which is then transformed into at least one ABP signal.

In another embodiment, an error function for determining a deviation between an ABP signal determined by transformation and a reference ABP signal is analyzed for generating the model, different signal portions of the ABP signal determined by transformation and/or the reference ABP signal and/or the deviation (the deviation signal) being weighted differently in the analyzis of the error function. Therefore, an ABP signal-specific error function can be used. The reference ABP signal may represent a basic truth and may be, for example, an ABP signal detected parallel to the input data (i.e., a heart-motion-induced signal) which was detected using a known, e.g., invasive ABP detection means. The error function is used to determine or quantify a deviation between the result of the transformation, i.e., the ABP signal determined by the transformation, and the basic truth. This deviation then influences the determination, particularly the training, of the model for the transformation by machine learning, particularly the determination of a neural network, the model being adapted so that, e.g., the deviation is reduced. Here, e.g., a mean square deviation or a mean absolute deviation may be determined as the deviation.

It is possible that, for the determination of the deviation, various signal portions of the ABP signal determined by transformation or the reference ABP signal are weighted differently, and all signal portions of the remaining signal are weighted identically. For the determination of the deviation, all signal portions of the ABP signal determined by transformation and all signal portions of the reference ABP signal are identically weighted, different portions of the signal representing the deviation, however, being weighted differently. A weighted portion in the deviation signal may be a portion chronologically corresponding to a predetermined (relevant) portion in the ABP signal determined by transformation and/or in the reference ABP signal.

The different weighting of different signal portions in at least one of the mentioned signals may advantageously improve a quality of the model and therefore also the signal quality of the ABP signal determined by transformation. The different weighting of different signal portions particularly renders a higher weighting of characteristic and therefore relevant portions of the ABP signal than of less relevant ones possible. Relevant ABP signal portions can be identified by an expert, for example by selecting signal portions using an input device. Alternatively, however, it is also conceivable to carry out an automated detection of relevant signal portions, for example using appropriate detection methods which, e.g., identify portions having predetermined signal properties. In such detection methods, for example, a phasor transformation may be carried out. In this case, predetermined weightings may be allocated to portions having predetermined signal properties. A relevant portion in a signal may be a systolic portion or a diastolic portion.

The systolic portion may be a time segment starting with an R-peak in an ECG signal recorded simultaneously with the ABP signal and ending at the point in time at which the T-wave following the R-peak ends. The diastolic portion may be a time segment starting at the end of a T-wave in an ECG signal recorded simultaneously with the ABP signal and ending at the point in time at which the R-peak following this end of the T-wave occurs. A period in an ABP signal may exhibit two local maxima, the first local maximum occurring first in time having a higher amplitude than the subsequent second local maximum. In this period of the ABP signal, the systolic portion may be a time segment staring briefly prior to the rise in blood pressure to the first local maximum and ending when the local minimum in between the two local maxima is reached. The diastolic portion starts thereafter and ends at the beginning of the next systolic portion.

In another embodiment, the transformation is carried out using a neural network. For example, the neural network may be implemented as an autoencoder, or as a convolutional neural network (CNN), or as an RNN (recurrent neural network), or as ab LSTM network (long short-term memory network), or as a neural transformer network, or as a combination of at least two of the mentioned networks. Such a neural network, particularly the neural network implemented as an autoencoder, may be trained with the aid of the above-described training data here, the implementation of the transformation of a detected heart-motion-induced signal into the ABP signal then being possible after the training.

Here, the implementation of the neural network as an autoencoder advantageously provides for that the computational effort required for the transformation is low so that the transformation can be reliably and rapidly carried out by embedded systems and portable end devices such as, e.g., cellular telephones in a simple manner.

The implementation as a CNN advantageously allows for a reduction of the complexity of the network and is therefore suitable for devices having a low computing capacity. This relates to both the training phase and the inference phase. It is also advantageous that the period of time required for the training is short in a CNN, particularly shorter than in LSTM networks which also require comparably higher computing capacities.

However, the implementation as an LSTM network has a particularly good suitability for the analyzis of time series because their architecture takes the relation to time-related dependencies into account. This advantageously results in a high quality of the transformation and of the ABP signal determined thereby.

In an alternative embodiment, the transformation is performed using a predetermined mathematic model or using a predetermined transformation function which may, for example, be predetermined by a user. Particularly, it is possible to appropriately parameterize mathematical models for transforming heart-motion-induced signals into ABP signals. This advantageously results in an alternative, reliable, and fast generation of ABP signals.

In another embodiment, the at least one heart-motion-induced signal is detected in a contactless manner. If a plurality of such signals is detected, exactly one, a plurality of but not all, or alternatively all signals may be detected in a contactless manner.

This and associated advantages were explained above.

In another embodiment, the at least one heart-motion-induced signal is filtered prior to the transformation, and then the filtered heart-motion-induced signal is transformed into an ABP signal. The filtering may particularly be a high or band pass or band stop filtering. An associated filter for carrying out the filtering may particularly be a Butterworth or polynomial filter. If the filtering is a high pass filtering a cutoff frequency of the high pass filter may be, for example, in a range of 5 Hz to 8 Hz to reliably reduce the effects of motion artefacts on the heart-motion-induced signal. If the filtering is a band pass filtering a first cutoff frequency may be, for example, in a range of 5 Hz (inclusively or exclusively) to 8 Hz (inclusively or exclusively), and another cutoff frequency may be in a range of 30 Hz (inclusively or exclusively) to 35 Hz (inclusively or exclusively) to likewise reliably reduce the effect of motion artefacts which are, for example, outside of the range of 8 Hz to 30 Hz. The filtering may particularly be carried out by Butterworth filters or polynomial filters. This advantageously results in a more precise determination of the ABP signal, particularly even when the patient moves during the detection of the heart-motion-induced signal.

In another embodiment, the at least one heart-motion-induced signal is generated by a detection means of a device. Detection means were already explained above. Here, the device refers to a unit comprising the detection means. For example, the device may be a mobile telephone or a tablet PC. However, of cause also other embodiments of such a device are conceivable. Further, the transformation is carried out by a calculating means of the device. In other words, the device comprises both the detection means and the calculating means. Here, a calculating means may be implemented as or comprise a microcontroller or an integrated circuit.

For example, it is possible to carry out the transformation or a partial transformation using a programmable or hard-wired component, particularly a chip (e.g. an ASIC, an FPGA). Such a component may then carry out the transformation on its own or as part of a system-in-package (SiP). It is also possible to directly integrate the means for carrying out the transformation in a sensor for detecting the heart-motion-induced signal (e.g., a MEMS acceleration sensor) or in another electronic component, e.g. as a SoC (system-on-a-chip).

This advantageously results in a centralized detection and generation of ABP signals, for example, on an end device, particularly a mobile end device.

Apart from the explained means for signal processing, the device may also comprise means for signal storage, means for signal transmission, and means for display. On the other hand, it may also be possible that the device comprises none of or not all of the explained means. In this case, the detected heart-motion-induced signal may be transmitted to another device which comprises one or a plurality of other means. The ABP signal generated in this way may therefore also be visualized, for example by a display means of the device. The ABP signal may also be stored, for example, by a memory means of the device. It is further possible to transmit the ABP signal from the device to an external system, for example, via an appropriate communication means of the device.

Alternatively, the heart-motion-induced signal is transmitted to a calculating means outside of the device from the detection means, the transformation being carried out by this calculating means outside of the device. The calculating means outside of the device may particularly be a server means, or the calculating means of another device.

In this case as well, the heart-motion-induced signal can be visualized, for example, by a display means of the device to which end the ABP signals determined by the transformation carried out by the calculating means outside of the device are retransmitted to the device. It is of course also possible to visualize the ABP signal determined in this way by a display means outside of the device. To this end, the ABP signal can be transmitted to the associated other device for display. Further, the ABP signal determined in this way may be stored or further processed, for example, by the storage or calculating means outside of the device or another storage or calculating means (outside of the device). Here, the calculating means outside of the device may be or form a server means of a network, particularly of the Internet. Particularly, the calculating means outside of the device may be part of a server means offering cloud-based services. The transmission to the calculating means outside of the device may take place in a wireless manner, for example, using appropriate transmission methods. However, it is of course also possible to configure the transmission in a wire-bound manner.

This advantageously results in that the calculating means of a device which also comprises the detection means is not overburdened by the transformation. Particularly, it is therefore possible to carry out the detection of the heart-motion-induced signal using devices providing for a comparably low computing capacity so that then an associated transformation and potentially a further processing can be carried out by other computing systems having an, in comparison, higher computing capacity.

In another embodiment, the at least one heart-motion-induced signal is generated by a detection means of a device, and the ABP signal determined by transformation is presented on a display means of the device or on an external display means, for example, a display means of another device. For example, it is possible that the heart-motion-induced signal is transmitted from the device to a calculating means outside of the device and that the transformation is carried out there, the ABP signal determined in this way then being transmitted to another device, for example, another cellular telephone, and then shown on its display means. The ABP signal may also be retransmitted to the device and shown by its display means. The ABP signals may also be displayed by a display in a browser, particularly if the calculating means outside of the device is a server means or part of it.

In this way, it is possible that a remote monitoring may be carried out on the basis of the ABP signals generated according to the invention.

In another embodiment, a functional test of a detection means is carried out prior to the transformation of the at least one heart-motion-induced signal, the heart-motion-induced signal only being transformed when operability is detected. Operability may be detected, for example, when the detection means generates a chronologically variable output signal. If a chronologically constant output signal is generated or the output signal does not deviate from a constant output signal by more than a predetermined amount an absence of operability may be detected. Alternatively, or cumulatively, operability may be detected when the output signal exhibits characteristics deviating from predetermined noise characteristics, particularly the characteristics of white noise, by more than a predetermined degree. If this is the case operability can be detected. If this is not the case, the absence of operability may be detected. An absence of operability may also be detected when a sampling rate of the output signal deviates from a target sampling rate and/or a quantification of the output signal deviates from permissible quantification values. In case of an absence of operability, no transformation can be carried out.

This advantageously results in that the transformation is only carried out when an operability of the detection means can be assumed. In this way, an energy consumption is reduced when carrying out the method.

Alternatively, or cumulatively, a signal quality of the detected signal is determined prior to the transformation of the at least one heart-motion-induced signal, the heart-motion-induced signal only being transformed when the signal quality is higher than or equal to a predetermined amount. A signal quality may be, for example, a signal to noise ratio or a quantity representing this ratio. If this ratio is larger than a predetermined amount, the transformation may be carried out. A signal quality may also be larger than or equal to a predetermined amount when a deviation between a predetermined reference signal curve and a detected signal curve in a portion of the heart-motion-induced signal is smaller than or equal to a predetermined amount. This may also be referred to as a so-called template comparison. Here, a classical signal shape of a heart-motion-induced signal, i.e. of the reference signal curve, may be determined and stored. Then, a deviation between the signal curve of the detected heart-motion-induced signal and the reference signal curve may be determined using methods known to the person skilled in the art.

A signal quality may also be determined using appropriate models such as, e.g., neural networks. Training data for such models may be generated by, e.g., a user, or in a (semi-) automated way allocating a quality standard representing the signal quality to a heart-motion-induced signal. This allocation may also be referred to as an annotation. Here, the heart-motion-induced signal constitutes the input data, and the quality standard constitutes the output data of the training data set. Such training data may particularly be generated by generating and annotating heart-motion-induced signals in various spatial positions of the detection means, particularly relative to the heart, at various SNRs, under various ambient conditions, in various states of movement of the patient, etc.

It is further possible that such a model, particularly a neural network, for determining the signal quality is also used for filtering the training data for determining the model for the transformation generated by machine learning. Here, therefore, only such heart-motion-induced signals are used as input data for the training of the model for the transformation for which the signal quality is higher than a predetermined value.

With the analyzis of the signal quality as a prerequisite for performing the transformation it can advantageously be ensured that a reliable and high-quality transformation is carried out.

It is also possible that, in addition to the signal quality, a quality-impairing cause is determined using appropriate models such as, e.g., neural networks. Training data for such models may be generated by, e.g., a user, or in (semi-)automated manner allocating the quality-impairing cause to a heart-motion-induced signal. This allocation can also be referred to as annotation. Here, the heart-motion-induced signal constitutes the input data, and the cause constitutes the output data of the training data set. Quality-impairing causes may be, for example, the presence of artefacts, the arrangement of the detection means in spatial positions adverse to the detection, particularly relative to the heart, and/or the presence of adverse ambient or movement conditions.

If a quality-impairing cause can be determined in this way the user may be informed of the cause, for example via a display means. In addition, the user may receive a recommendation for action to remedy the cause.

Further, alternatively or cumulatively, a location, i.e., a spatial position and/or orientation of the detection means relative to the heart is determined prior to the transformation of the at least one heart-motion-induced signal, the heart-motion-induced signal being only transformed when the location corresponds to a predetermined location or deviates from it by less than a predetermined amount. For example, it is possible that the heart-motion-induced signal will only exhibit predetermined signal characteristics when the location corresponds to a predetermined location or deviates from it by less than a predetermined amount. Therefore, signal characteristics of the heart-motion-induced signal can be determined and compared with the predetermined signal characteristics. If the deviation is smaller than a predetermined amount the location corresponds to the predetermined location or deviates from it by less than a predetermined amount.

It is also possible that the location is determined using appropriate models such as, e.g., neural networks. Training data for such models may be generated by, e.g., a user, or in a (semi-)automated manner allocating the location to a heart-motion-induced signal. This allocation may also be referred to as annotation. Here, the heart-motion-induced signal constitutes the input data, and the location constitutes the output data of the training data set. Such training data may particularly be generated by generating and correspondingly annotating heart-motion-induced signals in various spatial positions of the detection means, particularly relative to the heart. If a location can be determined the user can be informed of the location, particularly its correctness, for example via a display means. In addition, the user may receive a recommendation for action for changing the location when it deviates from the predetermined location by more than the predetermined amount.

With the determination of the location as a prerequisite for performing the transformation, it can advantageously be ensured that a reliable and high-quality transformation is carried out. For example, it can be avoided that a detection means for detecting the heart-motion-induced signal is arranged in an incorrect manner, for example, that an acceleration sensor does not rest on a surface of the body, and that therefore a quality of the ABP signal determined by the transformation is reduced.

It is conceivable that, for determining the operability and/or the signal quality and/or the location, the heart-motion-induced signals intended for transformation are analyzed, the signals being used for the transformation if an operability is detected and/or the signal quality is larger than or equal to the predetermined value and/or the location does not deviate from the predetermined location by more than a predetermined amount. Alternatively, the operability and/or the signal quality and/or the location may be determined on the basis of heart-motion-induced signals not intended for transformation, a further detection of the heart-motion-induced signal for the transformation then being carried out if operability is detected and/or the signal quality is larger than or equal to the predetermined value, and/or the location does not deviate from the predetermined location by more than a predetermined amount.

Alternatively, the signal quality and/or the arrangement of the detection means relative to the heart may also be determined by determining a vital parameter other than the SCG signal, e.g., a parameter representing a characteristic of respiration, and determining, depending on the parameter, whether the signal quality is larger than or equal to the predetermined value and/or whether the arrangement corresponds to a predetermined arrangement or deviates from it by less than a predetermined amount. To this end, e.g., a predetermined allocation of the parameter to the signal quality and/or the arrangement may be analyzed. In this way, for example, it can be ensured that the system is carried on the body and arranged in an appropriate position for detecting information relevant for an ABP signal.

For example, a (raw) data signal representing a characteristic of respiration may be transformed from a time range into a frequency range, for example using a Fast Fourier Transformation. Then, e.g., the signal energy E may be calculated, e.g., according to

$$E = {\sum }_{u R}^{o R} {A}^{2}$$

E representing the signal energy of the respiration range, and A representing the amplitude of the respective frequencies in the respiration range (e.g., 0.1 Hz to 0.6 Hz). The lower respiration frequency limit is designated by uR here, the upper respiration frequency limit is designated oR here.

Then, an absence of respiration may be detected if the signal energy is smaller than a predetermined threshold. If the signal energy is larger than or equal to the predetermined threshold a presence of respiration may be detected. In case of an absence of respiration, it can be assumed that the system is not carried on the body and that therefore the arrangement deviates from a predetermined arrangement by more than a predetermined amount. In this case, no transformation of the heart-motion-induced signal is carried out. This advantageously results in that the transformation is only carried out when a correct utilization of the detection means can be assumed.

The ABP signal may particularly be the ABP signal of a human, i.e., a signal for/in human medical applications. However, the method may also be applied to the generation of an ABP signal of an animal, i.e., to the generation of a signal for/in veterinary applications. For example, a particularly unobtrusive and non-invasive ABP detection in animals advantageously renders a considerable reduction of stress in animals possible on which an ABP signal is to be detected, e.g., for diagnostic purposes.

For example, a detection means may be integrated in a harness or chest strap applied to the animal. Therefore, such sensorics can be bought and applied by the animal keeper himself/herself. For example, an acceleration sensor in the harness/chest strap may detect the heart-motion-induced signals of the animal and render the explained transformation possible. Likewise, the method may be used to be deployed by veterinarians in routine examinations. Since animals usually only exhibit symptoms of a disease of the cardiovascular system at a very late time diagnoses can already be rendered possible in an early stage of such diseases in this way. Here, the veterinarian may detect an ABP of the animal in an easy manner by applying an appropriate detection means or a device including a detection means, e.g., a smartphone. In addition, this concept of examination can be applied to both pet fishes, e.g. koi, and horses and camels which is particularly interesting in the field of competitive sports involving such animals.

In the domain of farmed animals, medical monitoring is regularly only carried out on a reduced scale depending on the costs or effort, e.g. by a veterinarian diagnosing by cohort. However, ABP monitoring would also yield valuable information relating to animal welfare to the physician here (for example, with regard to productivity, health status, stress evaluation, early identification of bacterial infections such as streptococci). However, to date, ABP monitoring of individual animals using the common methods is extremely elaborate and expensive. The proposed method offers a cost-effective and easy possibility of monitoring, e.g., when the heart-motion-induced signal is detected in a contactless manner, e.g., by using radar sensors. Therefore, animals can be monitored in a contact-free and thus also hygienic manner. This monitoring would be conceivable for farmed animals such as pigs, ruminants, but also fish. The proposed method may also be made use of in animal research. It is also applicable in zoos and wildlife parks for ensuring the health of the animals while involving as little stress as possible.

The proposed method is advantageously applicable in a contactless manner. Another advantage is its easy application and the high availability. Likewise, advantageous is the possibility to apply the method to SCG detection means in hospital beds or beds in care facilities or even in the domestic environment.

Likewise, advantageous is the easy usability in rural areas in which there is often a lack of general physicians and particularly medical specialists. The proposed method can be easily and cost-effectively deployed for tele-medical applications in such a scenario.

Further, an existing device including a system capable of detecting a SCG signal 2, e.g., an acceleration sensor or a gyroscope can be enabled to carry out the proposed method by a software update. Therefore, the functionality provided for by the method can be retrofit on a multitude of devices which ensures a wide applicability of the method.

A further advantage is that an easy and reliable, long-term detection of precordial movements (SCG signals) is possible which will then also render the long-term and reliable determination of an ABP signal possible, particularly over a period of more than 24 hours. Likewise, advantageous is that the required sensorics are cost-effective, and required sensors are already installed in many usable devices and may therefore be used for implementing the method—as explained above.

Likewise, the proposed method is applicable to the subsequent transformation of already generated SCG signals 2 into ABP signals 1. This is particularly interesting in scientific studies.

Further proposed is a system for generating an ABP signal, the system comprising at least one detection means for detecting at least one heart-motion-induced signal and at least one calculating means. As explained above, the detection means and the calculating means may respectively be part of a device. However, it is also conceivable that the detection means and the at least one calculating means are respectively parts of devices different from each other. It is also conceivable that the system comprises a plurality of detection means for detecting a plurality of heart-motion-induced signals.

Further, the at least one detected heart-motion-induced signal is transformable into at least one ABP signal using the calculating means. For this purpose, it may be required to transmit the signal detected by the detection means to the calculating means, for example using transmission systems.

According to the invention, the transformation is carried out using a model generated by machine learning. Further, the heart-motion-induced signal constitutes the input value, and the ABP signal constitutes the output value of the transformation.

The system advantageously renders the implementation of a method for generating an ABP signal according to one of the embodiments described in this disclosure including the associated mentioned advantages possible. Therefore, the system is configured so that such a method can be carried out using the system.

In another embodiment, the detection means is integrated in an incubator. For example, the detection means may comprise a Doppler radar sensor and be arranged on a ceiling of the incubator in this case, particularly so that a chest area of the patient lying on a mattress of the incubator is arranged in the detection range of the radar sensor. Alternatively, the detection means may comprise or be implemented as an acceleration sensor disposed in/on the bottom of the incubator or in/on the mattress of the incubator.

Alternatively, the detection means may be arranged in a bed, particularly a hospital bed. If the detection means is implemented as, e.g., a Doppler radar sensor it may be arranged under the mattress or above the bed, for example, attached to a lifting pole.

Likewise, conceivable is the previously explained implementation of the detection means as an acceleration sensor which is arranged in/on the mattress or in/on the base of the bed. It is also possible to implement the detection means as pressure sensor which is arranged in/on the mattress of the bed.

Further, the detection means is alternatively integrated in a vehicle seat. Here, the detection means implemented as a Doppler radar sensor may be arranged, for example, in/on a seat backrest. A detection means implemented as a pressure sensor may be arranged in/on the seat backrest. The same applies to a detection means implemented as an acceleration sensor. Further, the detection means is alternatively integrated in a cardiac pacer. Further the detection means is alternatively integrated in a pet supply article, e.g., a chest strap, a halter, a collar, or the like.

Therefore, also a system for generating an ABP signal which comprises an incubator is described, the detection means being arranged in/on the incubator or in/on a mattress of the incubator. Further, a system for generating an ABP signal which comprises a bed is described, the detection means being arranged in/on the bed or in/on of a mattress of the bed. Further, a system for generating an ABP signal is described which additionally comprises a vehicle seat, the detection means being arranged in/on the vehicle seat. Further, a system for generating an ABP signal is described which additionally comprises a cardiac pacer, the detection means being arranged in/on the cardiac pacer. Further, a system for generating an ABP signal is described which additionally comprises a pet supply article, the detection means being arranged in/on the pet supply article. Of course, other applications are also conceivable. Also described is an incubator, a bed, a mattress, a vehicle seat, a cardiac pacer, and a pet supply article comprising at least the detection means of such a system.

Further, a computer program product comprising a computer program is proposed, the computer program comprising software means for performing one, a plurality of, or all steps of the method for generating an ABP signal according to one of the embodiments described in this disclosure if the computer program is executed by or in a computer or an automation system.

Further, a program is described which, when executed on a computer or in a automation system, causes the computer or the automation system to perform one or a plurality of or all steps of the method for generating an ABP signal according to one of the embodiments described in this disclosure, and/or a program storage medium on which the program is stored (particularly in a non-transitory form), and/or a computer comprising the program storage medium, and/or a (physical, e.g., electric, e.g., technically generated) signal wave, e.g., a digital signal wave which carries information representing the program, e.g., the abovementioned program which, e.g., comprises code means capable of performing one or all of the process steps described here.

This means that the method according to the invention is, for example, a computer implemented method. For example, all steps or only some of the steps (i.e., less than the entirety of the steps) of the method according to the invention may be carried out by a computer. An embodiment of the computer implemented method is a use of the computer for carrying out a data processing method. For example, the computer comprises at least one microcontroller or processor and, for example, at least one memory for (technically) processing the data, for example electronically and/or optically. For example, the processor is made of a substance or composition which is a semiconductor, for example, at least partly, an n- and/or p-doped semiconductor, for example, at least a II, III, IV, V, VI semiconductor material, for example (doped) silicon and/or gallium arsenide. The described steps, particularly the transformation, are carried out by, for example, a computer. Determination steps, calculation steps, or transformation steps are, for example, steps for determining data within the scope of the technical method, for example, within the scope of a program. A computer is, e.g., any type of data processing device, e.g., an electronic data processing device. A computer may be a device which is generally regarded as such, e.g., desktop PCs, notebooks, netbooks, etc., may, however, also be any programmable device such as, e.g., a mobile telephone or an embedded processor. For example, a computer may include a system (network) of “sub-computers”, each sub-computer representing a separate computer. Steps performed or carried out by a computer or an automation system may particularly be the determination steps and/or the verification step.

The computer program product advantageously renders the implementation of a method for generating an ABP signal according to one of the embodiments described in this disclosure possible the technical advantages of which were described above.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 shows a schematic illustration of a method for the determination of an ABP signal according to the invention.

FIG. 2 shows a schematic block diagram of a system for generating an ABP signal according to the invention according to of a first embodiment.

FIG. 3 shows a schematic illustration of a system for generating an ABP signal according to the invention according to another embodiment.

FIG. 4 shows a schematic flow diagram of a method according to the invention.

FIG. 5 shows a schematic illustration of a system for generating an ABP signal according to another embodiment.

FIG. 6 shows a schematic illustration of a system for generating an ABP signal according to another embodiment.

FIG. 7 shows a schematic illustration of a system for generating an ABP signal according to another embodiment.

FIG. 8 shows a schematic illustration of an example application of the method according to the invention.

FIG. 9 shows a schematic illustration of a system for generating an ABP signal including an incubator.

FIG. 10 shows a schematic illustration of a system for generating an ABP signal including a hospital bed.

FIG. 11 shows a schematic illustration of a system for generating an ABP signal including a vehicle seat.

FIG. 12 shows a schematic illustration of a method according to the invention in another embodiment.

FIG. 13 shows a schematic illustration of the generation/of the training of the neural network illustrated in FIG. 12.

FIG. 14 shows a schematic flow diagram of a method according to the invention in another embodiment.

FIG. 15a shows a schematic illustration of a dog strap including a detection means of a system for generating an ABP signal.

FIG. 15b shows a schematic illustration of a holster including a detection means of a system for generating an ABP signal.

FIG. 16 shows a schematic illustration of a pacemaker including a system for generating an ABP signal.

FIG. 17 shows an example illustration of weightings of different signal portions.

In the drawings, reference numbers may be reused to identify similar and/or identical elements.

### DETAILED DESCRIPTION

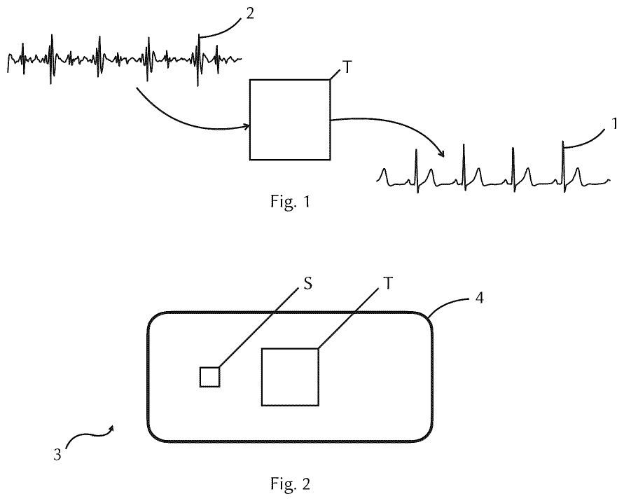

FIG. 1 shows a schematic illustration of a method for generating an ABP signal 1. Here, a heart-motion-induced signal embodied by an SCG signal 2 is detected. This may take place using an SCG detection means S explained in more detail in the following. Then, the detected SCG signal 2 is transformed into an ABP signal 1 by a transformation unit T, which may particularly be implemented as a calculating means or comprise a calculating means. Alternatively, or cumulatively, also a PCG signal may be detected as the heart-motion-induced signal and transformed into an ABP signal 1, e.g., by a PCG detection means. Further, alternatively or cumulatively, also a BCG signal may be detected as the heart-motion-induced signal and transformed into an ABP signal 1, e.g. by a BCG detection means.

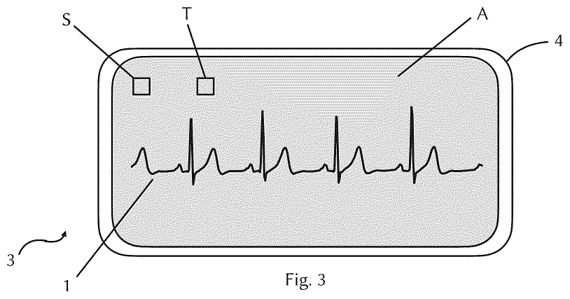

FIG. 2 shows a schematic block diagram of a system 3 for generating an ABP signal 1 (see FIG. 1). The system 3 comprises an SCG detection means S and at least one transformation unit T implemented as a calculating means. What is shown is that the SCG detection means and the transformation unit are part of a device 4, for example, a mobile telephone.

FIG. 3 shows an illustration of the system 3 for generating an ABP signal 1 according to another embodiment. As explained above, the system 3 comprises a SCG detection means S and a transformation unit T implemented as a calculating means. Further illustrated is a display means A on which the ABP signal 1 is visualized. Here, it is illustrated that the SCG detection means S, the transformation unit T, and the display means A are part of a device 4.

The SCG detection means illustrated in FIG. 2 and FIG. 3 may, for example, be implemented as an acceleration sensor, a pressure sensor, or a radar sensor, particularly a Doppler radar sensor or comprise such a sensor. Likewise, the SCG detection means may be implemented as a gyroscope or comprise such a gyroscope.

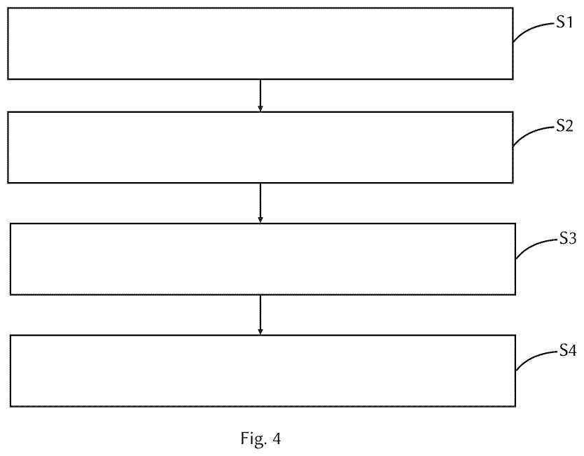

FIG. 4 shows a schematic flow diagram of a method according to the invention. Here, an SCG signal 1 is detected in a detection step S1, particularly using an SCG detection means S which was explained above. In an optional filtering step S2, the SCG signal 2 detected in this way is filtered, for example high pass filtered. A so-called detrending of the SCG signal 2 may also be carried out. In a transformation step S3 which may be carried out in the transformation unit T the SCG signal is transformed into an ABP signal. Therefore, also a seismocardiogram can be transformed into a continuous aortic blood pressure signal. The transformation step S3 may also comprise a plurality of partial transformations. In a post processing step S4, the ABP signal generated in this way, or the aortic blood pressure signal generated in this way is stored, transmitted to at least one other system, and/or visualized, for example on an appropriate display means A.

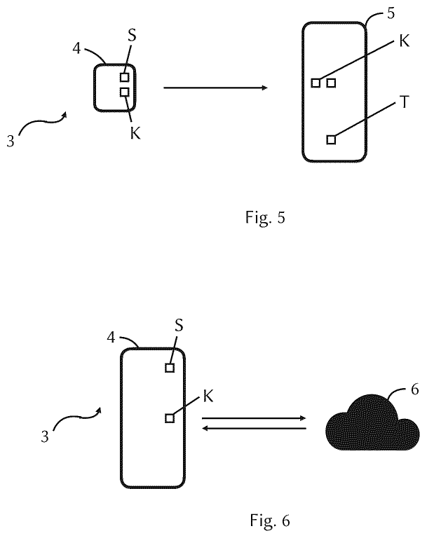

FIG. 5 shows a schematic illustration of a system 3 for generating an ABP signal 1 (see FIG. 1) according to another embodiment. Illustrated is a device 4 which comprises an SCG detection means S. With this SCG detection means S, an SCG signal 2 (see FIG. 1) is detectable. The device further comprises a communication means K for the data transmission between the device 4 and other devices. The generated SCG signal 1 is transmitted to a HUB device 5 by this communication means K. This HUB device 5 includes a transformation unit T implemented as a calculating means, and a communication means K for receiving the transmitted SCG signals. Further, the transformation of the SCG signal 2 into the ABP signal 1 may be carried out by the HUB means 5. It is then possible that the ABP signal 1 determined in this way is then displayed on a display means of the HUB means 5 which is not illustrated. It may also be stored by a memory means of the HUB means 5 which is not illustrated or further transmitted by the communication means K.

FIG. 6 shows another illustration of a system 3 for generating an ABP signal 1. In contrast to the embodiment illustrated in FIG. 5, the SCG signals 2 generated by the SCG detection means S are transmitted to a server means 6 offering so-called cloud-based services through the communication means K. This server means 6 may comprise a transformation unit T which is not illustrated which carries out the transformation of the SCG signals 2 transmitted from the device 4 into ABP signals 1. In FIG. 6, it is illustrated that the transformed signals, i.e., the ABP signals 1, are retransmitted to the device 4 so that they may then be received by the communication means K of the device 4. Then, the ABP signal received in this way may be stored, further processed, or visualized by the device 4, for example by a display means A of the device 4 which is not illustrated.

Here, it is possible that at least one post processing step is carried out by the HUB means 5 or by the server means 6. Here, individual, a plurality of but not all, or even all of the above-described post processing steps may be carried out by the HUB means 5 or the external server means 6.

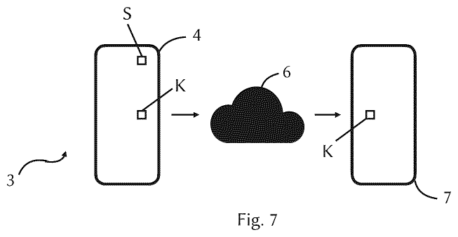

FIG. 7 shows a schematic illustration of a system 3 for generating an ABP signal 1 according to another embodiment of the invention. In contrast to the embodiment illustrated in FIG. 6, SCG signals 2 detected by the SCG detection means S of the device 4 are transmitted to the server means 6 the transformation unit of which will then carry out the transformation into an ABP signal 1 via the communication means K of the device 4. The ABP signal 1 determined by transformation in this way is then transmitted to another device 7 by the server means 6 and received using a communication means K of the other device 7 there. Further, the ABP signal 1 generated in this way may then be stored in a memory means of the other device 7, further processed by a calculating means of the other device 7, or displayed by a display means of the other device 7 which is not illustrated.

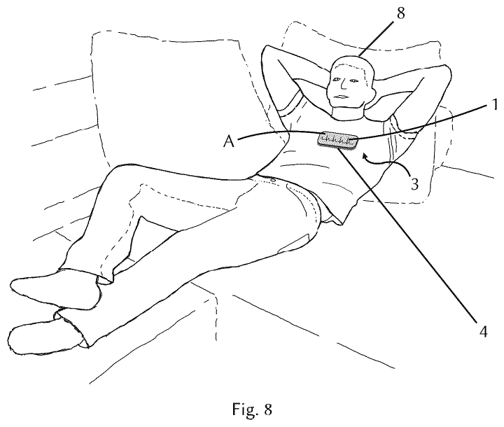

FIG. 8 shows a schematic application of a system 3 (see, e.g., FIG. 2) for generating an ABP signal 1. Here, a device implemented as a cellular telephone 4 which comprises a SCG detection means S which is not illustrated, and a transformation unit T implemented as a calculating means is placed on a chest of a user/patient 8. Of course, it is also conceivable that, instead of the cellular telephone 4, another device including a SCG detection means S is used. With the SCG detection means S, SCG signals 2 can then be generated which are then transformed into ABP signals 1 by the transformation unit (not illustrated) of the device 4 and then visualized by a display means A of the device 4.

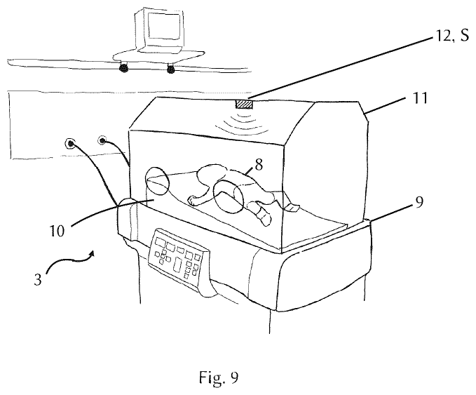

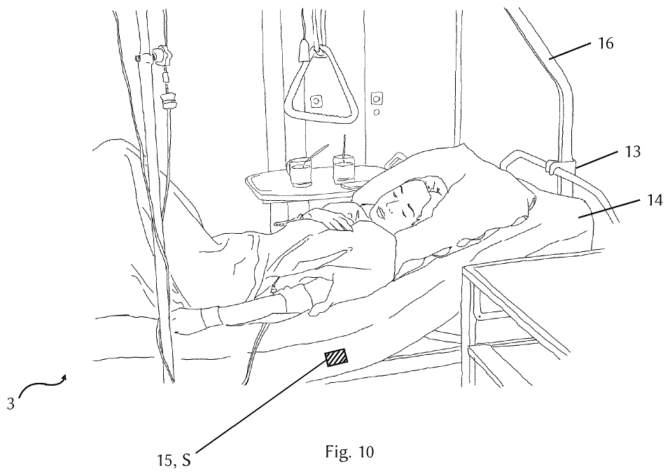

FIG. 9 shows an illustration of a system 3 for generating an ABP signal 1 (see FIG. 1) according to another embodiment. The system 3 comprises an incubator 9, a patient 8, for example a prematurely born infant, lying on a mattress 10 of the incubator 9. The incubator 9 further comprises a lid 11 covering the resting space for the patient 8. On the lid, an SCG detection means S implemented as a Doppler radar sensor 12 is arranged. Here, this Doppler radar sensor 12 is arranged so that a chest area of the patient 8 is located in the detection range of this sensor 12. Alternatively, it would be possible to dispose, e.g., an SCG detection means S implemented as a pressure or acceleration sensor in/on the mattress 10 or in/on a bottom of the incubator 9 on which the mattress 10 is supported. If the patient 8 is a prematurely born infant or a newborn infant an ABP signal 1 completely or to a large extent cleared of environmental artefacts can be generated, particularly using appropriate filtering methods since with the comparably high heart frequency of a newborn infant a reliable reduction of interfering influences of other persons in the surroundings of the incubator 9 can be achieved.

FIG. 10 shows a schematic illustration of a system 3 for generating an ABP signal 1 (see FIG. 1) according to another embodiment. The system 3 comprises a bed 13 including a mattress 14. The system 3 further comprises a SCG detection means S implemented as a pressure or acceleration sensor 15 which is arranged in/on the mattress 14. Of course, it is also conceivable to use a Doppler radar sensor which may, for example, be disposed on a lifting pole 16 of the bed 13.

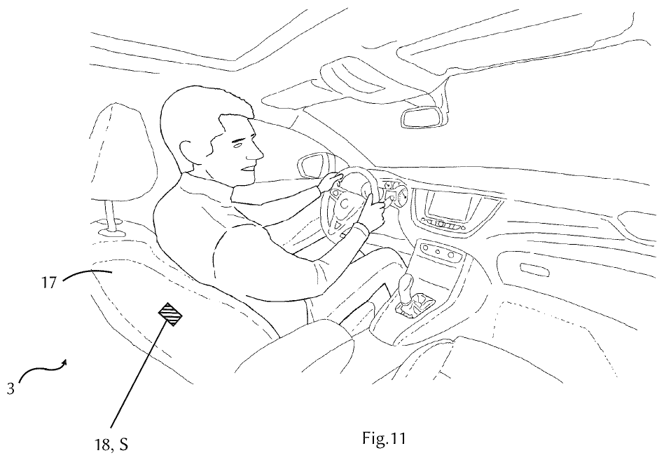

FIG. 11 shows a schematic illustration of a system 3 for generating an ABP signal 1 (see FIG. 1) according to another embodiment. Here, the system 3 comprises a vehicle seat 17, an SCG detection means S implemented as a pressure or acceleration sensor 18 being arranged in a back rest of the vehicle seat 17. Of course, it is also conceivable to implement the SCG detection means S as a Doppler radar sensor and to place in/on the back rest or in a position of the vehicle other than that in an appropriate way.

Apart from the normal monitoring of vital data, particularly of the blood pressure, and the normal, blood pressure-based diagnosis of pathologies, the embodiments illustrated in FIGS. 8, 9, 10, 11 render an inexpensive, continuous, as well as non-invasive monitoring and therefore also the detection of pathologies potentially not diagnosed so far, e.g., hypertension not diagnosed so far possible.

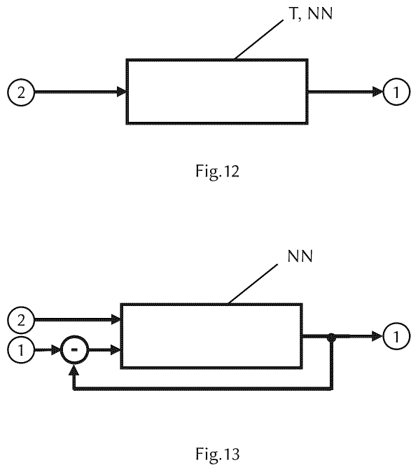

FIG. 12 shows a schematic illustration of a method according to the invention in another embodiment. Here, it is illustrated that SCG signals 2 constitute input data for a neural network NN which carries out the transformation of SCG signals into ABP signals 1. Therefore, the output signals of the neural network NN are the ABP signals 1 to be generated as proposed. In this case, the transformation unit T is implemented as or comprises a neural network NN or can perform functions of a neural network NN.

FIG. 13 shows a schematic illustration of the creation/training of the neural network NN illustrated in FIG. 12. In the process, training data in the form of simultaneously detected SCG signals 2 and ABP signals 1 are input into the neural network NN, parameters of the neural network NN being adapted so that a deviation of the ABP signals 1 generated by the neural network which are output data of the neural network NN from ABP signals of the training data set is minimized.

The training data set may result from a combined measurement of ABP signals and a seismocardiogram, i.e., SCG signals.

For generating training data, by way of example, continuous aortic blood pressure signals and seismocardiography signals were simultaneously recorded. For recording/detecting the SCG signals, the Shimmer3 ECG Unit, distributed by Shimmer Research Ltd. was used. This system renders the simultaneous detection of ECG signals and SCG signals possible. In parallel, an invasive clinical aortic blood pressure measurement for detecting an ABP signal was carried out, and a clinical surface ECG for detecting a further ECG signal was recorded by a clinical electrophysiology system by Phillips. The two detected ECG signals (the Shimmer3-ECG signal and the clinical surface ECG), particularly section I in the respective ECG signal was used for the synchronization of the SCG signals and the ABP signal. Then, remaining asynchronies were corrected, particularly with the aid of the so-called dynamic time warping (DTW) method or the automated, individual segment shift.

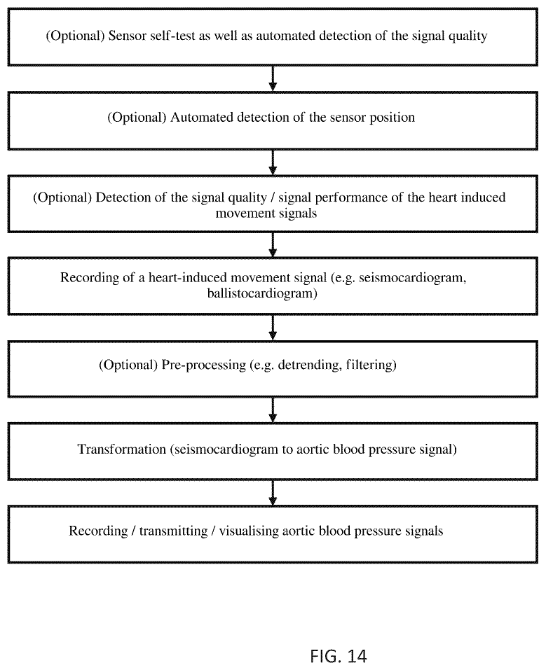

FIG. 14 shows a schematic flow diagram of a method according to the invention in another embodiment. In a first pre-detection step SOa, an operability of a detection means S for detecting a heart-motion-induced signal is determined. If it is given a signal quality of the signal detected by the detection means S is determined in a second pre-detection step SOb. If operability is not given the method is interrupted and, where appropriate, an error signal is output to a user.

If the signal quality is higher than a predetermined threshold a spatial position and/or orientation of the detection means S relative to the heart is determined in a third pre-detection step SOc. If the signal quality is not higher than the predetermined threshold the method is interrupted and, where appropriate, an error signal is output to a user. If the relative position does not deviate from a target relative position by more than a predetermined amount an SCG signal 1 is detected in a detection step S1 as already explained with reference to FIG. 4 above, particularly using an SCG detection means S which was explained above. The other steps S2, S3, S4 are identical to steps S2, S3, S4 illustrated in FIG. 4, therefore, the associated explanations are referred to. If the relative position deviates from a target relative position by more than a predetermined amount the method is interrupted and, where appropriate, an error signal is output to a user. A signal for repositioning may also be output to the user.

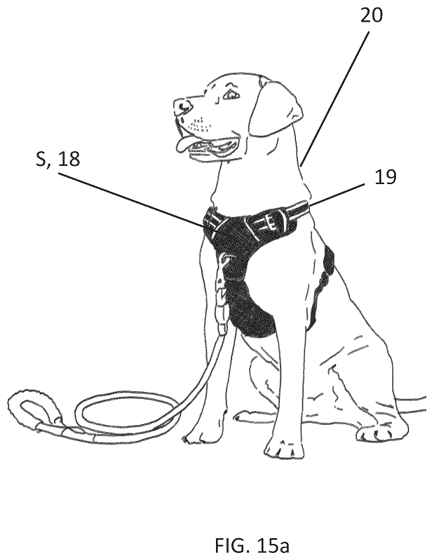

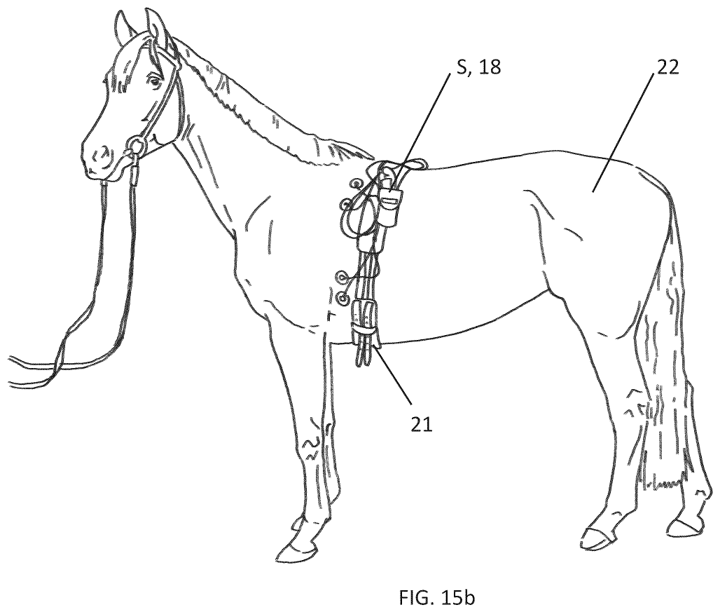

FIG. 15a shows a schematic illustration of a dog strap 19 including an SCG detection means S of a system 3 for generating an ABP signal 1 (see FIG. 1), the SCG detection means S being implemented as an acceleration sensor 18. It is illustrated that the SCG detection means S is arranged in a section of the dog strap 19 which abuts on a chest area of the dog 20 which wears the dog strap 19 as intended.

FIG. 15b shows a schematic illustration of a horse halter 21 including an SCG detection means S of a system 3 for generating an ABP signal 1 (see FIG. 1), the SCG detection means S being implemented as an acceleration sensor 18. It is illustrated that the SCG detection means S is arranged in a section of the halter 21 which abuts on an upper back area of the horse 22 which wears the halter 21 as intended. However, it is also conceivable to arrange the SCG detection means S in a section of the halter 21 which abuts on the abdominal or chest area of the horse 22 which wears the halter 21 as intended.

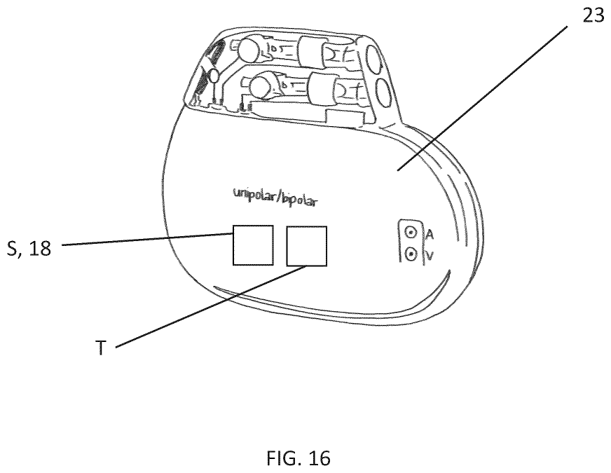

FIG. 16 shows a schematic illustration of a pacemaker 23 including a system 3 for generating an ABP signal 1. Illustrated is a rate adaptive cardiac pacer 23 comprising an SCG detection means S implemented as an acceleration sensor 18. The pacemaker 22 further comprises a transformation unit T. Not illustrated is a communication means K of the pacemaker 23 which can transmit the ABP signal 1 determined by transformation to a device outside of the body, e.g., a display means A or a server means 6. However, it is not mandatory that the pacemaker 23 comprises the transformation unit T. For example, it is also possible that the pacemaker 23 does not comprise a transformation unit T and that the output signals (raw signals) of the SCG detection means S are transmitted to a calculating means outside of the pacemaker, e.g. through the communication means K.

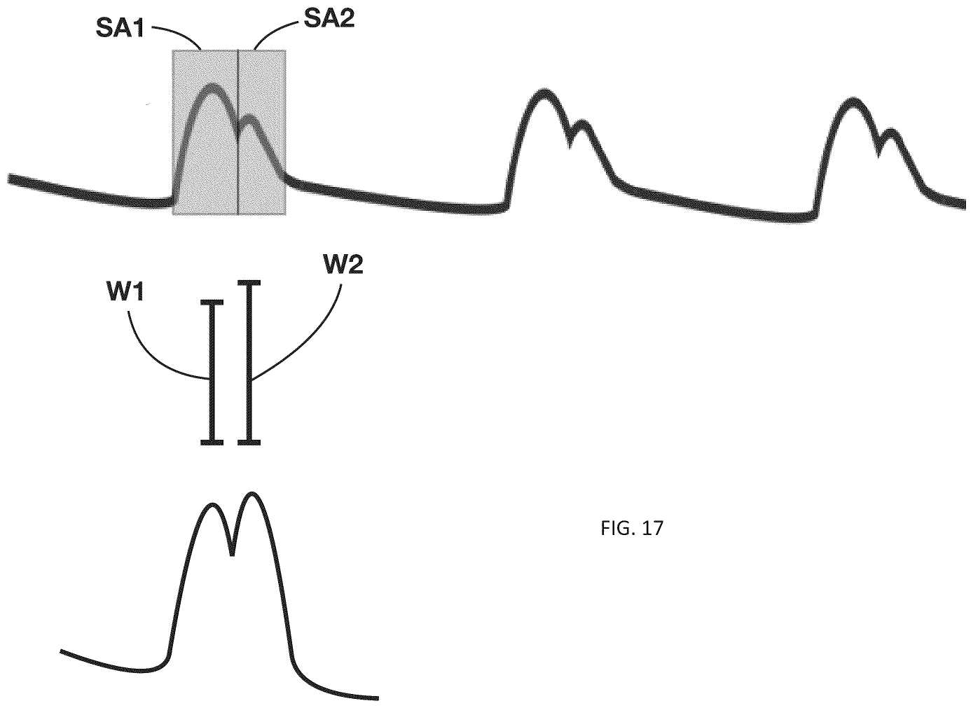

FIG. 17 shows an example illustration of weightings of various signal portions for the analyzis of an error function. In the upper line, an ABP signal is illustrated. In the ABP signal, two different signal portions SA1, SA2 are illustrated, the different signal portions being surrounded by a rectangle. The first signal portion SA1 is a systolic signal portion, and the second signal portion is a diastolic signal portion. The second, central line shows weighting factors w1, w2 allocated to the individual signal portions SA1, SA2. For example, a first weighting factor w1 is allocated to the first signal portion SA1, and a second weighting factor w2 to the second signal portion SA2. It can be seen that the first weighting factor w1 is smaller than the second weighting factor w2. It is possible that the weighting factors are larger than one. However, it is also possible that all weighting factors w1, w2 are equal to and larger than one so that the signal portions SA1, SA2 relevant for an ABP are weighted higher in relation to the remaining signal portions which are not relevant. The third, lower line shows a signal curve of the weighted ABP signal, the amplitude of the ABP signal in the first signal portion SA1 being weighted, particularly multiplied by the first weighting factor w1, and in the second signal portion SA2 by the second weighting factor w2. The weighting may also take place by a convolution of the ABP signal with a window function. With this weighting, particularly an amplitude compensation can be carried out. In this way, it can be avoided that significant signal changes are weighted higher than smaller changes which is the case, e.g., in the determination of the deviation using the method of the mean square error. However, in case of the ABP signal, small elevations (e.g., those in the signal curve framed in the first signal portion SA1) contain important information. It is conceivable that, in this way, different signal portions of an ABP signal 1 determined by transformation as well as different signal portions of a reference ABP signal are weighted and that then, after the weighting, the deviation between the weighted signals is determined to train the model for the transformation, particularly a neural network.

The term non-transitory computer-readable medium does not encompass transitory electrical or electromagnetic signals propagating through a medium (such as on a carrier wave). Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The term “set” means a grouping of one or more elements. The elements of a set do not necessarily need to have any characteristics in common or otherwise belong together. The phrase “at least one of A, B, and C” should be construed to mean a logical (A OR B OR C), using a non-exclusive logical OR, and should not be construed to mean “at least one of A, at least one of B, and at least one of C.” The phrase “at least one of A, B, or C” should be construed to mean a logical (A OR B OR C), using a non-exclusive logical OR.

1. A method for generating an arterial blood pressure (ABP) signal, the method comprising:
generating a model using machine learning;
detecting a heart-motion-induced signal; and
transforming the heart-motion-induced signal into the ABP signal by inputting the heart-motion-induced signal into the model and using an output of the model as the ABP signal.

2. The method of claim 1 wherein the heart-motion-induced signal is a seismocardiography (SCG) signal.

3. The method of claim 1 wherein the heart-motion-induced signal is a phonocardiography (PCG) signal.

4. The method of claim 1 wherein the heart-motion-induced signal is a ballistocardiography (BCG) signal.

5. The method of claim 1 wherein the model includes a neural network.

6. The method of claim 5 wherein the neural network is a convolutional neural network.

7. The method of claim 1 wherein:
generating the model includes analyzing an error function for determining a deviation between the ABP signal and a reference ABP signal; and
in analyzing the error function, different weightings are applied to different signal portions of at least one of the ABP signal, the reference ABP signal, or the deviation.

8. The method of claim 1 wherein the heart-motion-induced signal is detected in a contact-free manner.

9. The method of claim 1 further comprising:
filtering the heart-motion-induced signal to generate a filtered heart-motion-induced signal,
wherein the filtered heart-motion-induced signal is inputted into the model.

10. The method of claim 1 wherein:
the heart-motion-induced signal is generated by a detection means of a device; and
the transformation is carried out by at least one of:
a calculating means of the device, or
a calculating means of another device to which the heart-motion-induced signal is transmitted.

11. The method of claim 1 wherein:
the heart-motion-induced signal is generated by a detection means of a device, and
the ABP signal is displayed on at least one of:
a display means of the device, or
a display means of another device to which the heart-motion-induced signal is transmitted.

12. The method of claim 1 further comprising:
prior to the transformation of the heart-motion-induced signal, performing a functional test of a detection means,
wherein the heart-motion-induced signal is only transformed in response to the functional test indicating operability of the detection means.

13. The method of claim 1 further comprising:
prior to the transformation of the heart-motion-induced signal, determining a signal quality of the heart-motion-induced signal,
wherein the heart-motion-induced signal is only transformed in response to the signal quality being greater than or equal to a threshold value.

14. The method of claim 1 further comprising:
prior to the transformation of the heart-motion-induced signal, determining an arrangement of a detection means relative to a heart,
wherein the heart-motion-induced signal is only transformed in response to the arrangement deviating from a predetermined arrangement by less than a threshold amount.

15. The method of claim 1 wherein the heart-motion-induced signal is the only input value of the model.

16. The method of claim 1 wherein the ABP signal is a continuous ABP signal.

17. The method of claim 16 wherein the continuous ABP signal defines a blood pressure for each point in time of a predetermined determination period.

18. A system for generating an arterial blood pressure (ABP) signal, the system comprising:
detection means for detecting a heart-motion-induced signal; and
calculating means for transforming the heart-motion-induced signal into the ABP signal,
wherein the calculating means includes a model generated by machine learning,
wherein the heart-motion-induced signal constitutes an input value to the model, and
wherein the ABP signal constitutes an output value of the model.

19. The system of claim 18 wherein the detection means is integrated in at least one of an incubator, a bed, a vehicle seat, a cardiac pacer, or a pet supply article.

20. A non-transitory computer-readable medium comprising instructions including:
generating a model using machine learning;
detecting a heart-motion-induced signal; and
transforming the heart-motion-induced signal into an arterial blood pressure (ABP) signal by inputting the heart-motion-induced signal into the model and using an output of the model as the ABP signal.
